(19) United States
(12) Patent Application Publication
(10) Pub. No.: US 2026/0022338 A1
(43) Pub. Date: Jan. 22, 2026

(54) EDIBLE SCAFFOLDS FOR CULTURED MEAT PRODUCTION

(71) Applicant: NEXTURE BIO, INC., Wookland, CA (US)

(72) Inventors: Jed K. JOHNSON, Woodland, CA (US); Devan OHST, Woodland, CA (US); Hardy Z. CASTADA, Woodland, CA (US); Mitchell T. KAHN, Woodland, CA (US); Kevin DO, Woodland, CA (US); Marilyn MCNAMARA, Woodland, CA (US)

(73) Assignee: NEXTURE BIO, INC., Woodland, CA (US)

(21) Appl. No.: 18/993,926

(22) PCT Filed: Jul. 12, 2023
(86) PCT No.: PCT/US2023/070035

§ 371 Date: Jan. 13, 2025

Related U.S. Application Data
(60) Provisional application No. 63/388,489, filed on Jul. 12, 2022.

(51) Int. Cl. C12N 5/077

(52) U.S. Cl. CPC C12N 5/0652; C12N 2533/50; C12N 2533/74; C12N 2537/10

(57) ABSTRACT

An edible scaffold for cultured meat production. The scaffold comprises a polysaccharide and a plant-based protein formed of fibers, microcarriers, or other structural elements. The structural elements can comprise a diameter between about 1 μm to about 1000 μm. The scaffold can be formed via extrusion or an electrohydrodynamic technique. The scaffold further undergoes a surface modification treatment configured to facilitate attachment by cells. The scaffold can be used to culture myoblasts and other cells for the production of a meat product. The scaffold is incorporated into and ultimately a component of the resulting meat product.

### PRIORITY

The present application claims priority to U.S. Provisional Patent Application No. 63/388,489, titled EDIBLE SCAFFOLDS FOR CULTURED MEAT PRODUCTION, filed Jul. 12, 2022, which is hereby incorporated by reference herein in its entirety.

### BACKGROUND

The concept of lab-grown meat originally arose from space travel research. It was suggested that if meat could be grown in vitro, astronauts could grow their food to sustain longer space voyages. The idea was simple: culture mesenchymal stem cells into muscle, fat, and connective tissue to create an alternative to slaughtered meat. Since the concept was initially explored, several entities have begun researching and developing ways to commercialize cultured, or “clean,” meats. Motivations for this research include ideas of sustainability, animal welfare, carbon emissions, and consumer health.

Several companies have successfully developed cell culture methods to grow a product that includes muscle, fat, and/or connective tissue, but many of these products are limited to the traditional yields of a cell culture dish or flask. When most cells are cultured in a dish, for example, they form a monolayer, and the surface area of the layer is limited by the size of the dish or the number of cells. The cells in these cultures lack the necessary 3D environment to properly stack on top of one another, making it implausible to expect a noticeable volume or density increase from traditional cell culture techniques. This implausibility drastically affects the quality of and potential for cultured meat products to be grown under traditional methods. These cultured cells also generally lack the taste, structure, and texture of slaughtered meat. Therefore, there exists a need for the production of a thicker lab-cultured “clean” meat product with improved taste and texture.

Accordingly, 3D scaffolds have recently become of interest in cultured meat production. Meat product cells (e.g., myoblasts) can be cultured on such 3D scaffolds in order to create a correspondingly shaped and textured meat product that is more desirable than conventional meat products that are grown in culture dishes and the like. However, this introduces a new challenge; namely, conventional scaffolds are not edible and, therefore, the resulting meat product must be disentangled from the scaffold (e.g., by mechanically extracting the meat product from the scaffold or by dissolving the scaffold) before the meat product can be consumed. Disentangling the meat product from the scaffold can be costly and cumbersome, as well as potentially negatively affecting the properties of the cultured meat product itself. Therefore, it would be highly desirable for the scaffold for the cultured meat product to also be an edible product, thereby obviating the need to disentangle the cultured meat product from the scaffold.

Beneficially, the edible scaffolds could be formed from plant-based proteins, which would provide a host of other benefits, in addition to being edible (and already a food ingredient in many food products). Plant proteins are gaining popularity in tissue engineering and regenerative medicine applications. Plant protein-based scaffolds have been shown to possess desirable characteristics for the fabrication of biomaterials and more importantly, are biocompatible having less immunogenicity potential to support cell attachment, growth, proliferation, and differentiation. In addition to their biocompatibility and biodegradability, plant-based proteins are biochemically similar to the natural constituents of the extracellular matrix (ECM) with good mechanical properties. Their abundance in intra-and intermolecular disulfide bonds imparts plant proteins with their inherent water stability. Further, plant-based proteins and hydrolysates have concentrated balanced nutrients and are nutritionally complete which make them very attractive to serum-free cell/tissue culture. Despite the benefits, the use of plant-based proteins in edible scaffolds for cultured meat production has not gained significant traction as of yet.

Such edible scaffolds could take a variety of different forms, including fibrous scaffolds or microcarrier scaffolds. Further, the edible scaffolds could be constructed from hydrogels, which could be formed from alginate (an edible polysaccharide derived from brown algae), for example. Further, the edible scaffolds could be constructed using a variety of different techniques, including electrospinning, electrospraying, or extrusion.

Microcarrier scaffolds are matrices for anchorage-dependent cells that act as support structures for attachment and growth. These microcarriers can provide a highly effective surfaces for adherent cells to attach and grow, forming cell-microcarrier complexes leading to high expansion yield. Cellular adhesion involves the interaction of cell surface receptors or cell adhesion molecules with substrates on the surface of microcarriers and is enhanced by focal adhesion proteins and integrins. Microcarriers, with typical diameter of 100 to sub 500 μm, provide a high surface area to volume ratio for cell attachment and proliferation. Sufficient surface area is a critical parameter for culturing high-density populations of cells especially in large-scale production settings and has been found useful for tissue engineering and drug delivery applications.

A number of microcarriers with different types and properties have been developed commercially. Mainly, these microcarriers are classified according to their material composition (i.e., natural or synthetic), surface topography (i.e., smooth, micro-, or macroporous), the substance that cells grow on (i.e., solid or gel), and their shape or geometry (e.g., spherical or cylindrical). Commercial microcarriers also differ in terms of their surface coating/charge characteristics, surface chemistry (i.e., functional groups attached), stiffness, density, and stimulus responsiveness (i.e., pH, light, chemical, temperature, and electric/magnetic field) that could be used to trigger a specific cell behavior. In general, these different characteristics essentially determine cell attachment, proliferation, differentiation, and detachment. Despite the advancement in the development of microcarriers offering different characteristics for multiple cell types, commercial food-based or edible microcarriers have yet to be developed for the cell types (i.e., myoblasts) used in cultured meat production. In particular, microcarriers that are currently commercially available are typically made from polystyrene and are coated with animal-derived collagen or gelatin to assist in the cellular adhesion and otherwise facilitate cell growth and proliferation. However, such carriers pose at least two issues in cultured meat applications. First, polystyrene is not edible; therefore, the cultured cells need to be disentangled from the microcarrier scaffold before the meat product can be consumed. Second, the cultured meat industry is generally moving towards animal-component free (ACF) processes, which makes the animal derived collagen or gelatin coatings inapplicable with developing industry standards. In addition to the basic requirements for adhesion and proliferation of cells on microcarriers with sufficient mechanical properties, the scaffolds for meat production must be made from edible or food-based raw materials to meet food regulatory requirements. These edible microcarriers may then be included in the cultured meat product without additional modification or removal of the cells from the microcarriers.

Scaffolds are of particular interest in cultured meat production. Scaffolds provide three-dimensional (3D) structures for cells to attach to and proliferate along. Scaffolds facilitate the in vitro formation of more complex 3D structures like muscle fiber bundles, which allows the muscle tissue grown in bioreactors to more closely resemble muscle tissue organization found in animals. Scaffolds for cultured meat can take many forms. One popular group of scaffolds is known as hydrogels. This kind of scaffold is a gel made up of predominantly water held together by very hydrophilic filaments. These filaments can be crosslinked together to alter the hydrogel's properties and is commonly used to improve hydrogel scaffold stability. Hydrogels are attractive in cultured meat due to their low cost and ability to easily fill large volumes. Another major group of scaffolds used in cultured meat are solid scaffolds. These solid scaffolds are typically engineered to be substantially porous to allow for cell migration and nutrient exchange. Some popular methods of fabrication include, electrospinning, freeze drying, phase separation, self-assembly, extrusion, 3D printing, and more. These scaffold types allow for customization over physical structure, mechanical properties, and more all of which are used to optimize performance within the growth of animal tissues in vitro for cultured meat.

Therefore, there is a need in the prior art for edible scaffolds, particularly that incorporate plant-based proteins, for the production of cultured meat products.

### SUMMARY

The present disclosure is directed to cell culture scaffolds, particularly edible microcarrier-based cell culture scaffolds for cultured meat production.

In some embodiments, there is provided an edible scaffold for cultured meat production, the scaffold comprising: a structural element comprising a polysaccharide and a plant-based protein, wherein the structural element comprise a diameter between about 1 μm to about 1000 μm; wherein the structural element has been formed via extrusion or an electrohydrodynamic technique from a solution comprising the polysaccharide and the plant-based protein; wherein the structural element has undergone a surface modification treatment configured to facilitate attachment by cells, the surface modification treatment comprises at least one of a plant-derived polyphenolic compound, a cationic transition metal, or a plasma treatment; wherein the scaffold is formed in a shape configured to produce a meat product having a dimension from about 100 μm to about 500 mm when the cells are cultured thereon.

In some embodiments, there is provided a method of producing an edible scaffold for cultured meat production, the method comprising: depositing a solution comprising a polysaccharide and a plant-based protein into a cross-linking bath to form the edible scaffold comprising a structural element, the structural element comprising a polysaccharide and a plant-based protein, wherein the structural element comprise a diameter between about 1 μm to about 1000 μm; applying a surface modification treatment to the scaffold, the surface modification treatment configured to facilitate attachment by cells, the surface modification treatment comprises at least one of a plant-derived polyphenolic compound, a cationic transition metal, or a plasma treatment; wherein the scaffold is formed in a shape configured to produce a meat product having a dimension from about 100 μm to about 500 mm when the cells are cultured thereon.

In one embodiment of the edible scaffold or method, the structural element comprises a continuous fiber network.

In one embodiment of the edible scaffold or method, the structural element comprises microcarriers.

In one embodiment of the edible scaffold or method, the polysaccharide is configured to form a hydrogel.

In one embodiment of the edible scaffold or method, the polysaccharide comprises alginate.

In one embodiment of the edible scaffold or method, the electrohydrodynamic technique comprises at least one of electrospraying or electrospinning.

In one embodiment of the edible scaffold or method, the polysaccharide and the plant-based protein are present in a ratio from about 100:1 to about 1:20 by dry weight.

In one embodiment of the edible scaffold or method, the structural element is lyophilized.

In one embodiment of the edible scaffold or method, the polysaccharide and the protein are Generally Recognized as Safe (GRAS)-certified.

In one embodiment of the edible scaffold or method, the plant-based protein comprises at least one of a pea-based protein, soy-based protein, oat-based protein, mung bean-based protein, maize-based protein, chia-based protein, hemp protein, prolamin, pumpkin seed-based protein, rice-based protein, sunflower seed-based protein, or sacha inchi-based protein.

In one embodiment of the edible scaffold or method, the edible scaffold further comprises at least one of a food additive, a flavoring agent, a color additive, a preservative, a vitamin, a mineral, a nutritional additive or enhancer, a synthetic protein, a peptide, a ligand, or a cell culture media growth factor.

In one embodiment of the edible scaffold or method, the structural element is configured to undergo syneresis in response to a trigger.

In one embodiment of the edible scaffold or method, the trigger comprises a calcium chelator.

In one embodiment of the edible scaffold or method, the surface modification treatment is GRAS-certified.

In one embodiment of the edible scaffold or method, the surface modification treatment comprises iron citrate.

### FIGURES

FIG. 1 is a scanning electron microscope (SEM) image of a lyophilized microcarrier, in accordance with an embodiment of the present disclosure.

FIG. 2 is an SEM image of a lyophilized, polydopamine-coated microcarrier, in accordance with an embodiment of the present disclosure.

FIG. 3 is an SEM image of a lyophilized, tannic acid-coated microcarrier, in accordance with an embodiment of the present disclosure.

FIG. 4 is a light microscope image (10× magnification) of polydopamine-coated, non-lyophilized microbeads, in accordance with an embodiment of the present disclosure.

FIG. 5 is a light microscope image (10× magnification) of tannic acid-coated, non-lyophilized microbeads, in accordance with an embodiment of the present disclosure.

FIG. 6 is a light microscope image (40× magnification) of uncoated, lyophilized microbeads rehydrated in 1× phosphate buffered saline (PBS) solution, in accordance with an embodiment of the present disclosure.

FIG. 7 is a light microscope image (10× magnification) of polydopamine-coated, lyophilized microbeads rehydrated in 1× PBS solution, in accordance with an embodiment of the present disclosure.

FIG. 8 is a light microscope image (10× magnification) of tannic acid-coated, lyophilized microbeads rehydrated in 1× PBS solution, in accordance with an embodiment of the present disclosure.

FIG. 9 is a fluorescence microscope image of calcein-stained cultured cells seeded after 4 hours in a conical flask and grown in suspension showing cell viability and attachment to microcarriers that are surface treated with tannic acid/cationic transition metal coating, in accordance with an embodiment of the present disclosure.

FIG. 10 is an SEM image of a vacuum dried, 3D alginate hydrogel fibers scaffold, in accordance with an embodiment of the present disclosure.

FIG. 11 is an SEM image of a lyophilized, 3D alginate hydrogel fibers scaffold, in accordance with an embodiment of the present disclosure.

FIG. 12 is an SEM image of a vacuum dried, 3D alginate hydrogel fibers scaffold, in accordance with an embodiment of the present disclosure.

FIG. 13 is an SEM image of a vacuum dried, 3D alginate hydrogel fibers scaffold, in accordance with an embodiment of the present disclosure.

FIG. 14 is a light microscope image (40× magnification) of tannic acid-coated 3D alginate hydrogel fibers scaffold in water, in accordance with an embodiment of the present disclosure.

FIG. 15 is a light microscope image (40× magnification) of tannic acid-coated 3D alginate hydrogel fibers scaffold in water, in accordance with an embodiment of the present disclosure.

FIG. 16 is a light microscope image (100× magnification) of tannic acid-coated 3D alginate hydrogel fibers scaffold in water, in accordance with an embodiment of the present disclosure.

FIG. 17 is a light microscope image (100× magnification) of tannic acid-coated 3D alginate hydrogel fibers scaffold in water, in accordance with an embodiment of the present disclosure.

FIG. 18 is a light microscope image (100× magnification) of tannic acid-coated 3D alginate hydrogel fiber scaffold in water, in accordance with an embodiment of the present disclosure.

FIG. 19 is a light microscope image (100× magnification) of tannic acid-coated 3D alginate hydrogel fiber scaffold in water, in accordance with an embodiment of the present disclosure.

FIG. 20 is a fluorescence microscope image of a 3D alginate hydrogel fiber scaffold (4× magnification) stained with calcein following cell expansion, in accordance with an embodiment of the present disclosure.

FIG. 21 is a fluorescence microscope image of a 3D alginate hydrogel fiber scaffold (4× magnification) stained with calcein following cell expansion, in accordance with an embodiment of the present disclosure.

FIG. 22 is a fluorescence microscope image of a 3D alginate hydrogel fiber scaffold (10× magnification) stained with calcein following cell expansion, in accordance with an embodiment of the present disclosure.

### DETAILED DESCRIPTION

This disclosure is not limited to the particular systems, devices and methods described, as these may vary. The terminology used in the description is for the purpose of describing the particular versions or embodiments only, and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “pharmaceutical” is a reference to one or more pharmaceuticals and equivalents thereof known to those skilled in the art, and so forth.

As used herein, the term “about” means plus or minus 10% of the numerical value of the number with which it is being used. Therefore, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term “comprising” is used as the transition phrase, such embodiments can also be envisioned with replacement of the term “comprising” with the terms “consisting of” or “consisting essentially of.”

As used herein, the term “subject” includes, but is not limited to, humans and non-human vertebrates such as wild, domestic, and farm animals.

As used herein, the term “cultured meat product” means a meat product that is produced by human or machine intervention, rather than grown as a natural component of a living animal. A cultured meat product is thus not obtained directly from the slaughter of a living animal. Like traditional slaughtered meat, a cultured meat product is generally appropriate for consumption by one or more mammal species.

As used herein, the term “edible” means something that is suitable or safe to eat by a subject. “Edible” can include consumable products that are certified as Generally Recognized as Safe (GRAS) by the United States Food and Drug Administration (USFDA).

### Edible Cell Culture Scaffolds

The present disclosure generally relates to cell culture scaffolds, particularly edible cell culture scaffolds for cultured meat production. In use, the various embodiments of scaffolds described herein can be used as a medium on which cells (e.g., myoblasts) can be grown (e.g., in a bioreactor) to form a cultured meat product. Edible scaffolds are of particular interest in cultured meat production because if the meat product cells are cultured on a non-edible scaffold, then the meat product must be removed from the scaffold or the scaffold needs to be dissolved to ready the meat product for consumption. Processes for extricating the meat product from the scaffold on which it was cultured can be cumbersome and time-consuming, as well as potentially negatively affecting the properties of the cultured meat product. Scaffolds can serve multiple functions in the production of a cultured meat product. In particular, scaffolds provide three-dimensional (3D) structures for cells to attach to and migrate along. Further, scaffolds facilitate the in vitro formation of more complex 3D structures (e.g., muscle fiber bundles), which allows the cultured meat product grown in bioreactors to more closely resemble the structure of muscle tissue found in animals. In cultured meat production applications, the scaffolds on which the cells are grown further must themselves be edible because they become a part of the final cultured meat product. In other words, as the cells grow on the scaffold medium, the scaffold itself becomes integrated into and inextricable from the resulting cultured meat product.

Further, the scaffolds described herein can be designed to mimic the mechanical integrity, flexibility, ability to support cellular behavior, and nutrient composition of the ECM found in vivo, which acts as a support structure for muscle cells. Accordingly, the meat product scaffolds can be tuned so that the meat product grows in a manner that mimics the textural properties of conventional meat. For example, the stiffness of hydrogel scaffolds could be tuned to imitate the nonrigid characteristics of the different ECM components, which are important factors for cell behavior and final texture, thereby allowing the meat product to grow in a manner that mimics the mechanical properties of natural meat. Further, the scaffold structure may promote cellular attachment and induce the cells to differentiate into fibrous bundles that are similar to muscle fibers, thereby further mimicking the composition of the natural muscle tissue found in animals.

The scaffolds described herein could consist of a variety of different structural elements, including microcarriers, continuous fibers, or combinations thereof. Both microcarriers and fibers can be formed via extrusion or electrohydrodynamic (e.g., electrospinning or electrospraying) techniques, as described further below. Regardless of whether the scaffolds are constructed from microcarriers or continuous fibers, the scaffolds form a supporting substrate that cells can adhere to and proliferate on.

Microcarriers are small particles that collectively form a matrix when maintained within, for example, a suspension. In one embodiment, the microcarriers can include microbeads. In one embodiment, the microcarriers can include micronized fiber fragments, which are described further below. In some embodiments, the microcarriers could be less than 900 μm in diameter. The microcarriers can be, for example, 100-500 μm in diameter. The microcarrier scaffolds can be suspended within a bioreactor for growing cells therein, for example. In some embodiments, the scaffolds could include solid and/or fibrous scaffolds.

The scaffolds can be fabricated using a variety of different techniques and from a variety of different materials. In particular, the cell culture scaffolds can be both fabricated from materials that are edible and using techniques that render the fabricated scaffold product edible. In some embodiments, hydrogel scaffolds could be utilized for cultured meat production. Hydrogels are gels made predominantly of water held together by hydrophilic filaments. The hydrogels' properties can be altered by cross-linking the filaments, which is commonly used to improve the stability of the hydrogel scaffold. Hydrogels are particularly attractive in cultured meat product applications due to their low cost and ability to fill relatively large volumes. In other embodiments, solid scaffolds could be utilized for cultured meat production. Solid scaffolds may be engineered to be substantially porous to allow for cells to migrate into and throughout the scaffold and facilitate nutrient exchange.

In sum, the present disclosure describes edible scaffolds suitable for use in the production of cultured meat products. These scaffolds can be fabricated using a variety of different techniques, including electrospinning, electrospraying, freeze drying, phase separation, self-assembly, extrusion, or 3D printing, as described in greater detail below. Further, the physical structure, mechanical properties, chemical properties, and other such properties of the scaffolds can be customized in order to optimize the in vitro growth performance of the cultured meat product. The present disclosure further describes embodiments of these scaffolds that are fabricated from a combination of plant-based protein and polysaccharide hydrogel, in addition to other non-animal derived substances and additives. These scaffolds described herein are suitable for cell culture and tissue engineering in the production of cultured meat products.

### Fabrication Techniques for Edible Cell Culture Scaffolds

The scaffolds described herein can be fabricated using a variety of different techniques, including electrohydrodynamic (EHD) techniques and non-EHD techniques. EHD techniques can include, for example, electrospraying and electrospinning. Non-EHD techniques can include, for example, freeze drying, phase separation, self-assembly, extrusion, and 3D printing. The scaffolds described herein could be made using any of these techniques.

The fabrication techniques described herein are adapted to produce microcarriers that can be collectively used (e.g., in a suspension) to form a cell culture scaffold. In some embodiments, the microcarriers could include microbeads. Microbeads can be produced via, for example, the electrospraying techniques described below. In some embodiments, the microcarriers could include fiber fragments. Fiber fragments can be produced via, for example, the electrospinning techniques described below, in combination with a micronization processing step. The microcarriers can further be combined with a carrier medium in order to form a suspension. In some non-limiting examples, the carrier medium could include PBS, cell culture media, platelet-rich plasma, plasma, lactated Ringer's solution, a gel, a powder, an aerosol, or any combination thereof. When held in suspension, the microcarriers collectively form a matrix or scaffold that can support the growth, proliferation, and migration of cells thereon.

In some embodiments, the scaffolds can be fabricated at a constant temperature (e.g., room temperature or 25° C.). In some embodiments, the scaffolds can be fabricated at a constant relative humidity (e.g., 25%). Some of these fabrications are described in greater detail below. Embodiments of the scaffolds described can be fabricated using a variety of these different fabrication techniques.

### Electrospraying

Electrospraying (also referred to as “electrohydrodynamic atomization”) is a liquid atomization-based technique that utilizes an electric field to produce droplets or particles from a solution, which are deposited into a bath solution. In electrospraying, an injection system is used to gradually expel a solution into a bath as a high voltage is applied to the solution as it is expelled. The injection system may be similar to or different from the injection system described above for electrospinning. In one embodiment, an electrospinning system could include a high-voltage power source, a syringe capped with a metallic needle tip, a syringe pump capable of providing a variable flow rate (e.g., 0-50 mL/hr), and an aqueous bath solution containing crosslinking divalent ions (e.g., Ca²⁺). In some embodiments, the aqueous bath solution could include a variety of different compounds to provide the calcium ions, such as CaCl₂, CaC₆H₁₀O₆, CaSO₄, Ca₃(C₆H₅O₇)₂, CaCO₃, Ca₃(PO₄)₂), or combinations thereof. In some embodiments, the aqueous bath solution could further include food-grade accelerating agents, such as C₆H₁₀O₆, C₆H₁₀O₄, C₆H₈O₇, or combinations thereof. In some embodiments, the aqueous bath solution could further include chelating agents, such as Na₆P₆O₁₈, Na₄P₂O₇, Na₃C₆H₅O₇, CaH₄P₂O₈, or combinations thereof. In electrospraying systems, the solution expelled by the injection system is charged due to the high voltage being applied at the needle tip. As the charged solution flows, it breaks up into droplets and forms spherical particles or microbeads having a generally narrow size distribution as the droplets hit the aqueous bath solution. In some embodiments, the crosslinking bath solution may be charged with a high voltage. The spherical microbeads may continue to form into spherical or bead shapes as the gel further solidifies and stabilizes in the crosslinking solution. In some embodiments, the polymer injection system may move with respect to the receiving surface.

In some embodiments, the microbeads produced via electrospraying may have a diameter<500 μm. The characteristics of the microbeads (e.g., size and morphology) are affected by the concentration (e.g., a hydrogel-forming polysaccharide and a plant-based protein, as described further below), mixing ratio, and/or shear viscosity of the components of the electrospraying solution, as well as various electrospraying parameters, including the needle tip size, flow rate, electric potential difference, distance between the needle tip and crosslinking solution, temperature, humidity, and so on. Therefore, some or all of these characteristics of the polymer solution and/or the electrospraying system could be controlled or adjusted in order to control the resulting physical or chemical characteristics of the scaffolds.

### Extrusion

Extrusion is a technique of pressing material through a die to form a structure (e.g., a scaffold). The size and/or shape of the die can be altered in order to change the corresponding size, shape, thickness, and other properties of the extruded structure. Accordingly, extrusion can be utilized to form either continuous fibers or microcarriers. There are a variety of different forms of extrusion, including warm extrusion where the extruded solution is warmed prior to being pressed through the die, cold extrusion where the extruded solution is pressed through the die at room temperature, microextrusion which is extrusion performed at the submillimeter range, direct extrusion where the material is pressed by a ram through the die, and indirect extrusion where the die is stationary while container and die move. The present disclosure encompasses all of the various forms of extrusion.

Extrusion is generally performed by selecting a die having a desired cross-sectional arrangement (e.g., size or shape) for the solution (e.g., a solution comprising a blend of alginate and plant-based protein, as described above) being extruded. In direct extrusion, the extrusion system further includes a ram or syringe and a pump. In some implementations, the solution is extruded into an aqueous bath containing cross-linking divalent ions (e.g., Ca²⁺) prior to ejection. Calcium ions can be supplied by a solution of calcium chloride, calcium sulfate, calcium phosphate, calcium carbonate, or other divalent cationic compounds/salts that may contain food-grade accelerating agents (e.g., C₆H₁₀O₆, C₆H₁₀O₄, or C₆H₈O₇) and chelating agents (e.g., Na₆P₆O₁₈, Na₄P₂O₇, Na₃C₆H₅O₇, or CaH₄P₂O₈).

As the polysaccharide-protein solution is extruded into the cross-linking bath, the structural elements (e.g., continuous fibers or microcarriers) are formed from the extruded solution. In embodiments where fibers are extruded into the bath, the fibers become entangled, forming a web of micronized filaments. This tangled web of micronized filaments forms the scaffold. As the web of micronized filaments stabilizes in the cross-linking bath, the scaffold will further solidify and maintain its shape. In one embodiment, the scaffold can be formed into a cylindrical shape. Moreover, these fiber-like structures will have a narrow size distribution (i.e., will have consistent diameters within a particular tolerance). In one embodiment, the fibers can have diameters<500 μm. The properties of the extruded fibers (e.g., size and morphology) are affected by the concentration of the polysaccharide and protein, the mixing ratio and shear viscosity of the solution, and various extrusion parameters (e.g., needle tip size, flow rate, temperature, or humidity). Accordingly, any of these characteristics can be adjusted in order to tune the resulting properties of the scaffold as desired. In various embodiments, the fibers can also be formed in either a random orientation or an aligned orientation depending on the desired application. The alignment of the fibers can likewise affect the resulting properties of the scaffold, which thereby provides the ability to further tune the scaffold depending on the desired application.

### Surface Modification

Either during or subsequent to the fabrication processes described above, the scaffolds can undergo various different types of surface modification treatments. Surface modification techniques can encompass both physical and chemical-based surface modifications. Surface modification of the microcarriers that make up the scaffolds can be beneficial for a number of different reasons. For example, surface modification of the microbeads can create particular chemical moieties and/or a particular physical environment that encourages adhesion of the cultured cells to the scaffold, proliferation and migration of the cells throughout the scaffold, and integration of the cultured meat product with the scaffold.

In one embodiment, the surface treatment could include a polydopamine (PDA) coating. PDA is an extensively repeated residue of 3,4-dihydroxy-L-phenylalanine (dopamine) and lysine units with catechol and amine groups. In dopamine polymerization at an alkaline pH, the catechol moieties oxidize to quinone. The PDA coating can express latent reactivity to certain nucleophiles because of the amine and thiol groups that can be covalently conjugated on a PDA layer through the quinone moiety. A PDA coating on the scaffold can be beneficial because it provides a modified bio-surface having multiple functional groups and motifs that promote cell-adhesion properties, stimulate positive cellular responses, and can render biomimetic and favorable microenvironments that promote cell and tissue functions. Since cell adhesion is a critical preliminary step in tissue engineering, it plays a significant role in different cellular functions such as spreading, proliferation, migration, and differentiation. Microcarriers with surface properties that promote cell adhesion are especially beneficial for large-scale bioprocessing and production of cultured meat through maximizing and obtaining high yields of cells from small culture volumes.

In one embodiment, the surface treatment could include a tannic acid coating. Tannic acid is a USFDA GRAS-certified polyphenolic tannin consisting of a glucose core and surrounded by covalently conjugated galloyl units (3,4,5-trihydroxybenzoic acid) through an ester bond. The oligomerization of tannic acid is induced via oxidation reaction at a neutral pH (i.e., pH 7) and can form a colorless coating. Tannic acid can readily bind with organic and inorganic surfaces through covalent and non-covalent interactions. The multiple hydroxyl groups in each tannic acid molecule facilitates hydrogen bonding interactions. The negatively charged tannic acid coating at neutral pH, which is largely contributed by the galloyl moieties, permits its interaction with cationic molecules through electrostatic interactions. Moreover, non-polar interactions of tannic acid with hydrophobic molecules are stabilized by its multiple aromatic rings Tannic acid coating modifies the surface of the scaffold, which contributes to cell adhesion, spreading, and long-term culture. A tannic acid coating on the microcarriers can be beneficial because it can result in rapid cell anchorage and cell proliferation rates. Further, the tannic-coated, functionalized surface also favors cell spreading and reduces cell apoptosis. Ultimately, similar to PDA-coated scaffolds, tannic acid-coated microcarriers provide good cell adhesion surface properties that is critical for large-scale bioprocessing and production of cultured meat.

In one embodiment, the surface treatment could include a plasma treatment. In plasma treatment, the scaffolds are exposed to radiofrequency (RF) energy, which causes plasma to be generated from air or other gasses. Plasma modification introduces different chemical groups onto the surfaces of the microcarriers, thereby causing changes in the surface properties of the microcarriers. The changes in the surface properties could improve cell-material interactions. Surface properties such as wettability, energy, and roughness can also be affected by introducing functional groups thereto through plasma treatment.

In one embodiment, the surface treatment could include the application of a cationic transition metal. The cationic transition metal could include Cu⁺, Au⁺, Hg₂²⁺, Ag⁺, Cd²⁺, Cr²⁺, Co²⁺, Cu²⁺, Fe²⁺, Pb²⁺, Mn²⁺, Hg²⁺, Ni²⁺, Pt²⁺, Sn²⁺, Zn²⁺, Cr³⁺, Co³⁺, Au³⁺, Fe³⁺, Pb⁴⁺, Sn⁴⁺, or any combination thereof. In one embodiment, the cationic transition metal could be provided via treatment with, for example, iron citrate.

In one embodiment, the various embodiments of scaffolds described above could be treated with functional biological molecules. In particular, functional biological molecules can be adsorbed onto the surface of the scaffold's fibers, microcarriers, and/or other structural components. For example, the functional biological molecules could be adsorbed to the structural components' surfaces by exposing the microcarriers to a concentrated solution of the biological molecule. In some embodiments, the functional biological molecules could include arginylglycylaspartic acid, fibroblast growth factor, fibrin, recombinant collagen, or combinations thereof.

In some embodiments, the scaffold could undergo two or more types of surface modification treatments. For example, the scaffold could be treated with a tannic acid coating and further undergo the application of a cationic transition metal before, after, or in conjunction with the application of the tannic acid coating.

As generally described throughout, it is beneficial for the scaffold itself to be edible or otherwise biologically compatible in order to obviate the need to have the cultured meat product removed from the scaffold on which it is grown before consumption, as is conventionally done in the field. Therefore, in some embodiments, the surface modification treatment could be GRAS-certified or utilize GRAS-certified components. For example, the cationic transition metal could be applied via iron citrate, which is GRAS-certified. Some conventional scaffold surface treatment techniques deposit cationic transition metals via iron (III) chloride hexahydrate, iron (II) sulfate, or iron (III) sulfate, which are not GRAS-certified and thus not suitable for the applications described herein.

### Edible Scaffolds for Cultured Meat Production

As noted above, a variety of different techniques can be used to fabricate cell cultured scaffolds that are specifically adapted for use in cultured meat production. Further, these scaffolds can be fabricated from edible materials, which allows for them to be used in the production of cultured meat products. Further, these scaffolds can be subjected to various surface modification processes in order to encourage cellular integration and migration. Still further, these scaffolds can include any combination of the additives described above. The edible scaffolds described herein provide a non-animal-derived, edible substrate suitable for enterprise scale cell culture of cultured meat products.

In some embodiments, the scaffolds can be formed from a combination of a polysaccharide and a plant-based protein. In an embodiment where the scaffold is formed via electrospraying, the polysaccharide and the plant-based protein could be blended into a solution, which can then be electrosprayed to form the scaffold. Similarly, in an embodiment where the scaffold is formed via extrusion, the polysaccharide and the plant-based protein could be blended into a solution, which could then be extruded through an appropriately sized and/or shaped die (e.g., into a cross-linking bath) to form the scaffold. The polysaccharide and the plant-based protein could be edible or GRAS-certified. Plant protein-based scaffolds can be beneficial because they can possess desirable characteristics for the fabrication of biomaterials and are biocompatible and have less immunogenicity potential to support cell attachment, growth, proliferation, and differentiation. In addition to their biocompatibility and biodegradability, plant-based proteins are biochemically similar to the natural constituents of the ECM and provide similar mechanical properties as the ECM. Further, plant-based proteins have an abundance of intra-and intermolecular disulfide bonds, which makes them inherently stable in water (thereby allowing many such proteins to form hydrogels) Still further, plant-based proteins and hydrolysates are nutritionally balanced and complete, which makes them very attractive to serum-free cell/tissue culture.

In one illustrative embodiment, the polysaccharide could include alginate. In some embodiments, the polysaccharide could include a plant-derived polysaccharide that is configured to form a hydrogel, such as cellulose, pectin, exudate gums, hemicellulose, starch, and inulin. In some embodiments, the polysaccharide could include an algal-derived polysaccharide that is configured to form a hydrogel, such as alginate, agar, carrageenans, ulvan (from sea lettuces), and fucoidan. In some embodiments, the polysaccharide could include a microbial-derived polysaccharide that is configured to form a hydrogel, such as polysaccharides derived from bacterial fermentation sources (e.g., xantham gum, welan gum, and gellan gum) and fungal fermentation sources (e.g., Aureobasidium pullulans). In some embodiments, the polysaccharide could include an animal-derived polysaccharide that is configured to form a hydrogel, such as chitin, chitosan, heparin, chondroitin sulfate, and hyaluronic acid.

As used herein, the plant-based proteins described herein can include isolates, concentrates, hydrolysates, micronized proteins, functionalized proteins, solubilized proteins, base-treated proteins, and/or enzyme-hydrolyzed proteins. In some embodiments, the plant-based proteins could include pea-based proteins, soy-based proteins, oat-based proteins (e.g., avenin), mung bean-based proteins, maize-based proteins (e.g., zein), chia-based proteins, hemp-based proteins, prolamins (e.g., gliadin, hordein, secalin, or kafirin), pumpkin seed-based proteins, rice-based proteins, sunflower seed-based proteins, or sacha inchi-based proteins, ECM-derived proteins (e.g., fibronectin, laminin, collagen, vitronectin, or elastin), and any other edible, plant-based proteins. It should be noted that, as used herein, “plant-based” proteins include any proteins that are isolated, concentrated, hydrolyzed, micronized, functionalized, solubilized, base-treated, enzyme-hydrolyzed, or otherwise developed from a plant. For example, a “pea-based protein” would include any protein that is isolated, concentrated, hydrolyzed, micronized, functionalized, solubilized, base-treated, enzyme-hydrolyzed, or otherwise developed from a pea plant.

In one embodiment, the hydrogel-forming polysaccharide and the plant-based protein could be present in about the same amount by dry weight (e.g., the polysaccharide and the plant-based protein can be present in a 1:1 ratio by weight). In another embodiment, the hydrogel-forming polysaccharide and the plant-based protein could be present in different amounts (e.g., the polysaccharide and the plant-based protein can be present in a 2:3 ratio by weight). In some embodiments, the polysaccharide and the plant-based protein are present in a ratio from about 100:1 to about 1:20 by weight. In some embodiments, the scaffolds could include plant-based peptides in licu of or in addition to the plant-based proteins. For example, the peptides could include FCM-derived peptides (e.g., RGD, YIGSR, IKVAV, REDV, DGEA, KQAGDV, or VAPG).

In some embodiments, the scaffolds can be fabricated using any of the techniques described above, including both electrohydrodynamic techniques (e.g., electrospraying and electrospinning) and non-electrohydrodynamic technique (e.g., extrusion). In some embodiments, the microcarriers can undergo additional processing steps, such as the application of the surface treatment techniques described above.

Some of the properties of the scaffolds described herein can further be controlled by inducing or controlling the amount of crosslinking between the plant-based proteins and/or between the plant-based proteins and the other components of the scaffolds (e.g., the hydrogel-forming polysaccharide). The amount or degree of crosslinking by and/or between the plant-based proteins can be initiated by heat, pressure, change in pH, irradiation, and various other crosslinking techniques. In some embodiments, the solution from which the microcarriers are fabricated could include a crosslinking agent configured to induce crosslinking by and between the plant-based proteins. The crosslinking agent can include, for example, divalent cations (e.g., calcium ions), citric acid, ascorbic acid, phytic acid, or combinations thereof. In some embodiments, the scaffolds can be exposed to ultraviolet light to induce crosslinking by and between the plant-based proteins.

In one embodiment, the structural components (e.g., fibers or microcarriers) of the scaffolds may have a diameter or other dimension from about 100 to about 500 μm. In one embodiment, the structural components (e.g., fibers or microcarriers) of the scaffolds may have a diameter or other dimension from about 100 to about 900 μm. In one embodiment, the structural components (e.g., fibers or microcarriers) of the scaffolds may have a diameter or other dimension from about 100 to about 1000 μm. In some embodiments, the microcarriers may have a dimension of, for example, about 100 μm, about 150 μm, about 200 μm, about 250 μm, about 300 μm, about 350 μm, about 400 μm, about 450 μm, about 500 μm, about 600 μm, about 700 μm, about 800 μm, about 900 μm, about 1000 μm, or ranges between any two of these values, including endpoints. In one embodiment, the scaffolds could be fabricated from structural components having approximately the same dimensional range. In another embodiment, the scaffolds could be fabricated from structural components having different dimensional ranges. For example, the scaffolds could include a first microcarrier having a first diameter range and a second microcarrier having a second diameter range. The first and second microcarriers in this embodiment could include the same or different materials. In another embodiment, the scaffolds could be fabricated from structural components having the same dimensional ranges. For example, the scaffolds could include a first microcarrier having a particular diameter range and a second microcarrier having the same diameter range. The first and second microcarriers in this embodiment could include the same or different materials.

In some embodiments, the scaffolds could be formed into a variety of different structures and/or shapes. For example, the scaffolds could be shaped into the form of a steak or otherwise shaped such that the resulting cultured meat product is in the form of a steak. Further, the scaffolds could have sufficient mechanical properties to support a cultured meat product as it is grown from the cells seeded on the scaffolds. In certain embodiments, the cultured meat product may have a dimension (i.e., length, width, and/or thickness) from about 100 μm to about 500 mm. The dimension may be, for example, about 100 μm, about 200 μm, about 300 μm, about 400 μm, about 500 μm, about 600 μm, about 700 μm, about 800 μm, about 900 μm, about 1 mm, about 5 mm, about 10 mm, about 25 mm, about 50 mm, about 75 mm, about 100 mm, about 125 mm, about 150 mm, about 175 mm, about 200 mm, about 225 mm, about 250 mm, about 275 mm, about 300 mm, about 325 mm, about 350 mm, about 375 mm, about 400 mm, about 425 mm, about 450 mm, about 475 mm, about 500 mm, or any range between any two of these values, including endpoints. The scaffold can accordingly be configured to (e.g., have a sufficient size and/or shape) to produce a meat product having the aforementioned dimension(s) when the cells are cultured thereon.

In some embodiments, the scaffolds could include a variety of different additives, including food additives, flavoring agents, color additives, preservatives, vitamins, minerals, nutritional additives/enhancers, synthetic proteins, peptides, ligands, cell culture media growth factors, or combinations thereof. In some embodiments, the additives could be incorporated into the solution from which the scaffolds are fabricated (e.g., electrospun, electrosprayed, or extruded). In other embodiments, the additives could be applied to the scaffolds via spraying, sprinkling, soaking, or other surface treatments.

In one embodiment, the polysaccharide could be present from about 0.75 wt % to about 7.5 wt % in the solution. In various embodiments, the polysaccharide could be present in about 0.75 wt %, 1.0 wt %, 2.0 wt %, 3.0 wt %, 4.0 wt %, 5.0 wt %, 6.0 wt %, 7.0 wt %, 7.5 wt %, or any ranges between any two of these values, including endpoints. In one embodiment, the protein could be present from about 0.75 wt % to about 7.5 wt % in the solution. In various embodiments, the protein could be present in about 0.75 wt %, 1.0 wt %, 2.0 wt %, 3.0 wt %, 4.0 wt %, 5.0 wt %, 6.0 wt %, 7.0 wt %, 7.5 wt %, or any ranges between any two of these values, including endpoints. In one embodiment, any additives could be present from 1.0 wt % or less in the solution.

In some embodiments, the scaffolds could be subjected to any of the surface treatment techniques described above. In one embodiment, the surface modification treatment could be configured to facilitate cell attachment to the microbeads of the scaffold.

In some embodiments, the scaffolds can be lyophilized. Lyophilization is a mild drying process and results in causes the scaffolds to form a sponge-like configuration with a textured and/or porous structure. This embodiment can be beneficial because a porous structure for the scaffold can provide improved cell migration, attachment, growth, and vascularization. In particular, porous scaffolds having highly interconnected pores could efficiently improve the transport of nutrients and cellular metabolic wastes throughout the scaffolds, thereby resulting in improved cellular growth.

In some embodiments, the scaffold can be further configured to dissolve or undergo syneresis in response to a trigger (e.g., a sequestrant). For example, the trigger could include a calcium chelator. In this embodiment, a calcium chelator could be introduced to the cell culture media in order to pull the calcium ion from the crosslinked alginate, thereby causing the calcium-alginate hydrogel structure to collapse and cause syneresis (where liquid, such as water, is expelled or extracted from a gel). This would allow the scaffold to be removed from the resulting cultured meat product, which can be beneficial in certain circumstances.

In sum, the present disclosure describes a variety of different embodiments of scaffolds that improve the throughput and quality of cultured meat products. The various fabrication techniques described herein can produce edible, plant-based, non-animal derived scaffolds suitable for cultured meat production at larger scales than are currently possible in the cultured meat technical field, without the need to further modify or remove the cells from the microcarriers. Further, the fabrication techniques described herein can be used to create scaffolds that are edible and are derived from non-animal sources.

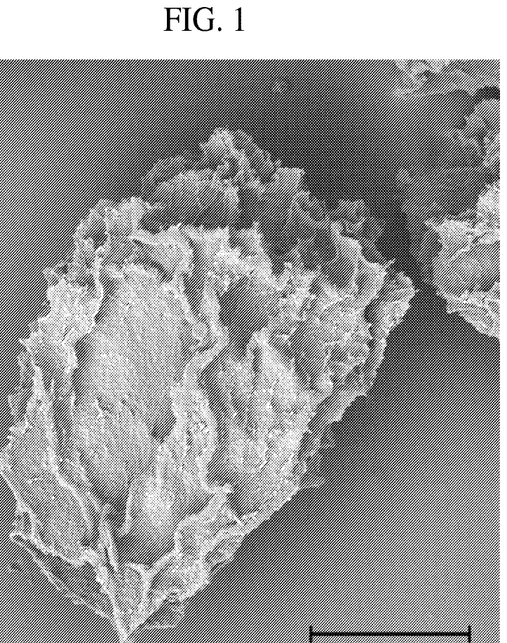

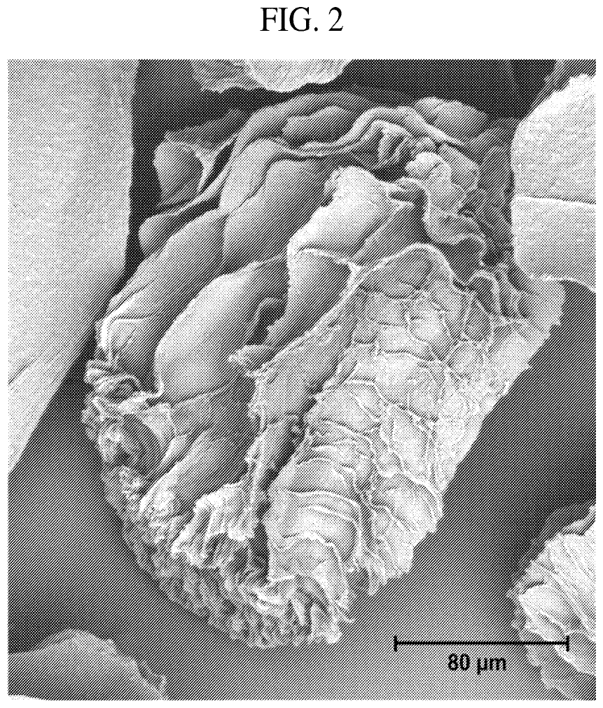

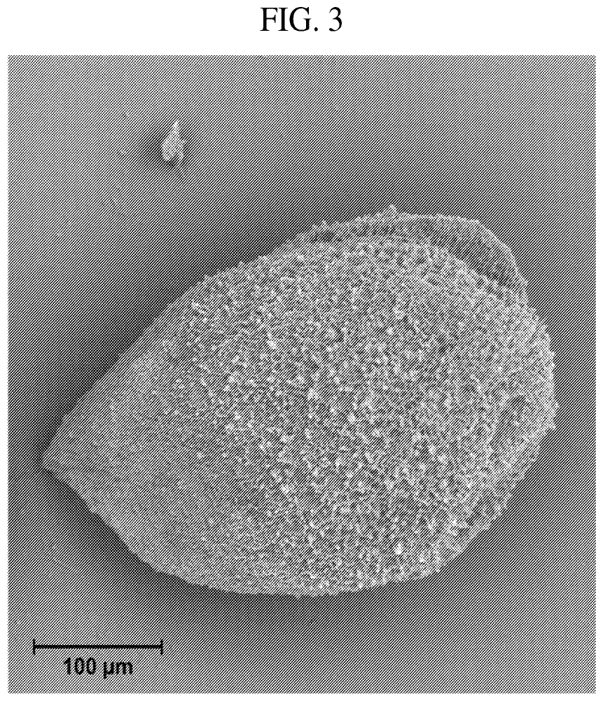

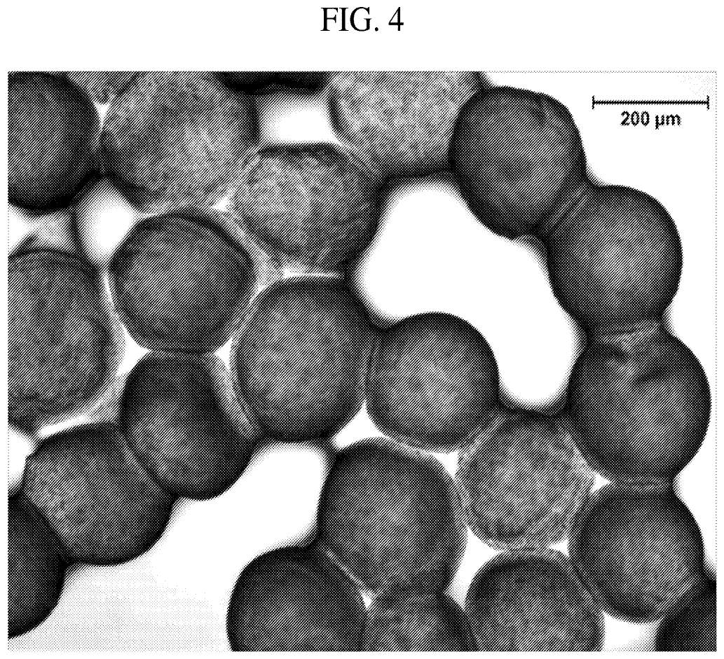

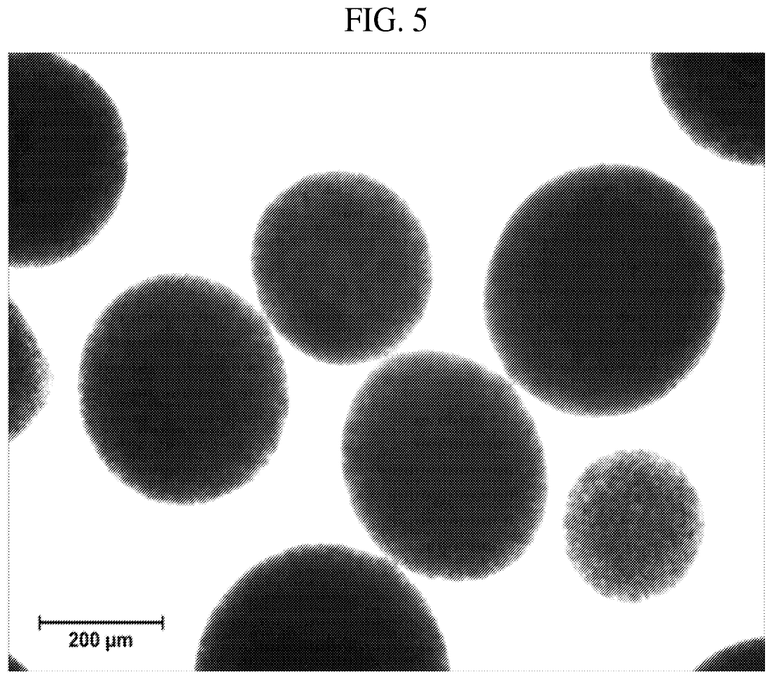

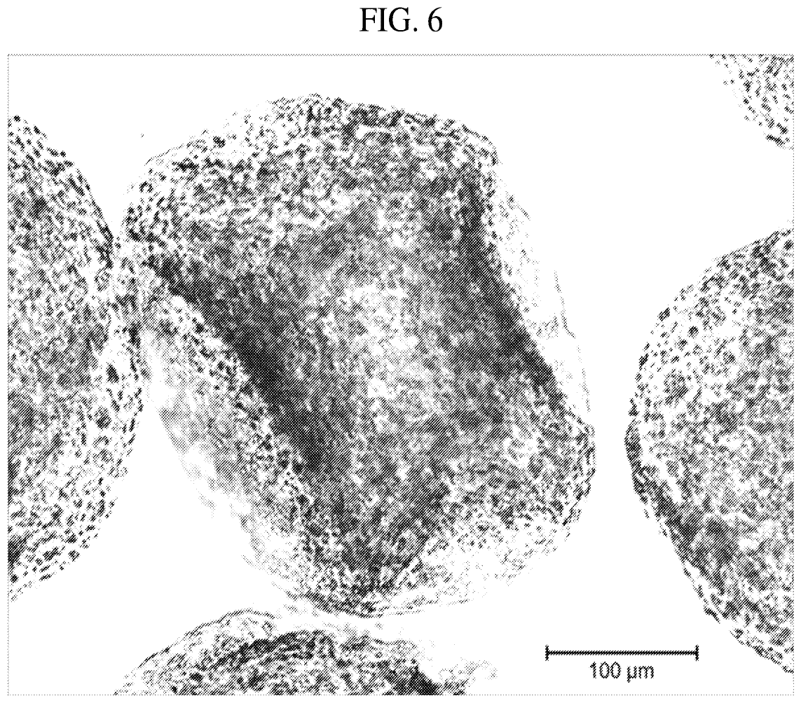

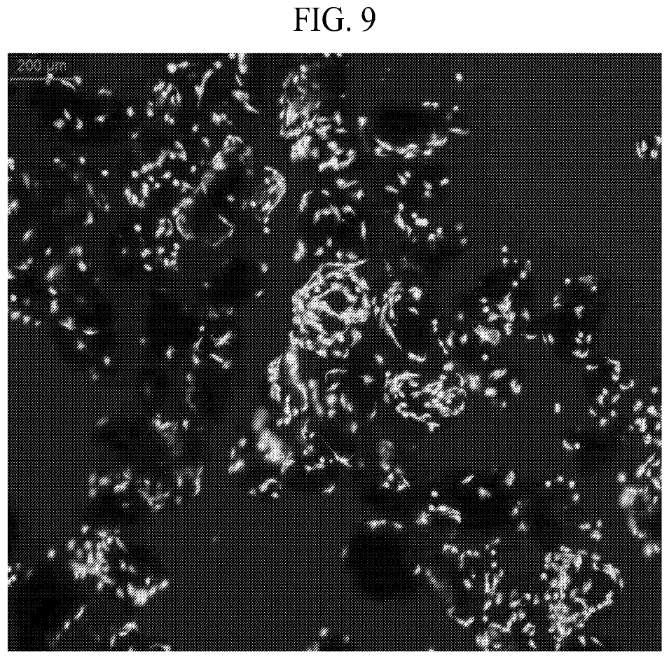

As noted throughout the present disclosure, the scaffolds can, in some embodiments, comprise microcarrier scaffolds. To further illustrate these techniques, some specific embodiments of microcarriers fabricated using the techniques described above to have different combinations of features are shown in FIGS. 1-9. In particular, FIGS. 1-3 and 6-8 show lyophilized microcarriers and FIGS. 4 and 5 show non-lyophilized microcarriers. FIGS. 2, 4, and 7 show microcarriers that have undergone surface modification with a PDA coating. FIGS. 3, 5, and 8 show microcarriers that have undergone surface modification with a tannic acid coating FIG. 6 shows microcarriers that have not undergone any surface modification. Further, FIGS. 6-8 show microcarriers that have been combined with PBS to form a suspension. Further, the microcarrier scaffold embodiments described herein have been experimentally confirmed to encourage cell proliferation and growth. For example, FIG. 9 shows a fluorescence microscope image of calcein-stained cultured cells seeded after 4 hours in a conical flask and agitated in an orbital shaker showing cell viability and attachment to microcarriers that are surface treated with tannic acid/cationic transition metal coating. Accordingly, the electrosprayed microcarrier scaffolds described herein can be used to culture cells for the manufacture of a cultured meat product. Various embodiments of the microcarrier scaffolds could include any other combination of features described herein.

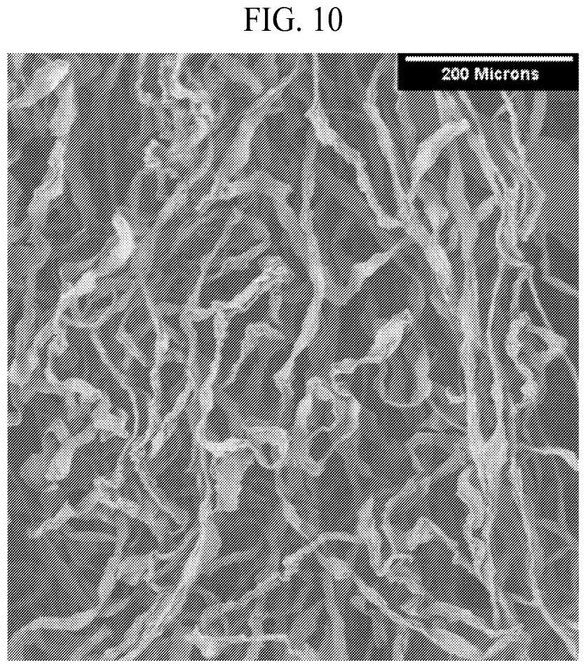

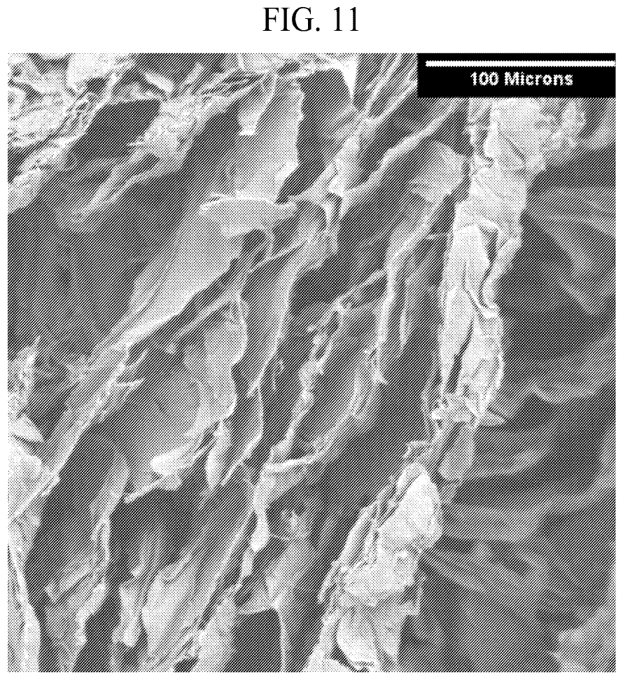

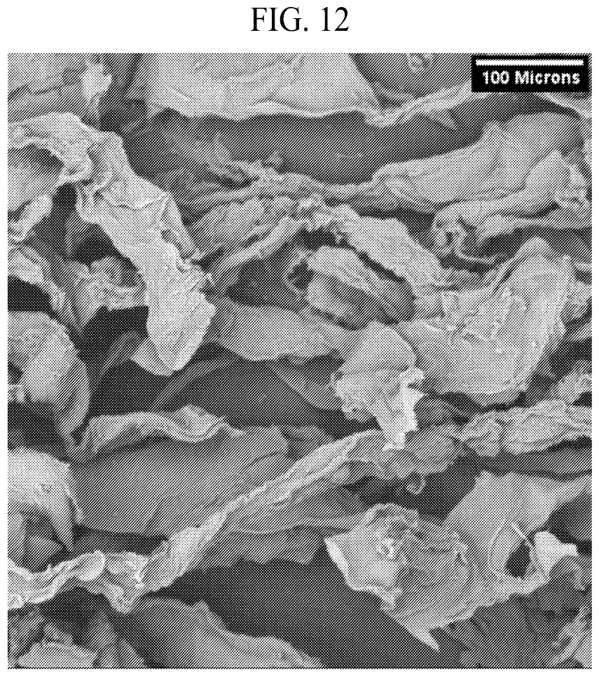

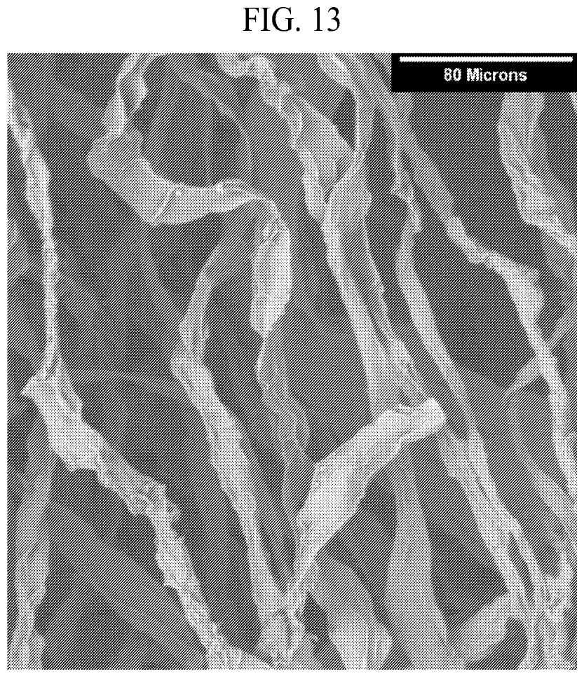

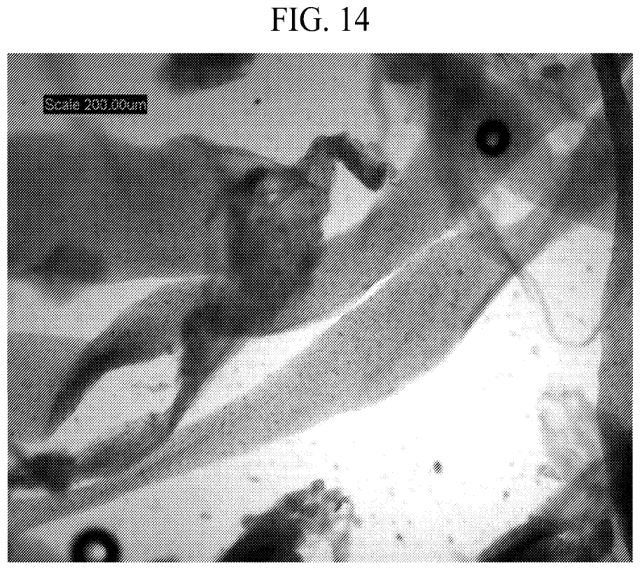

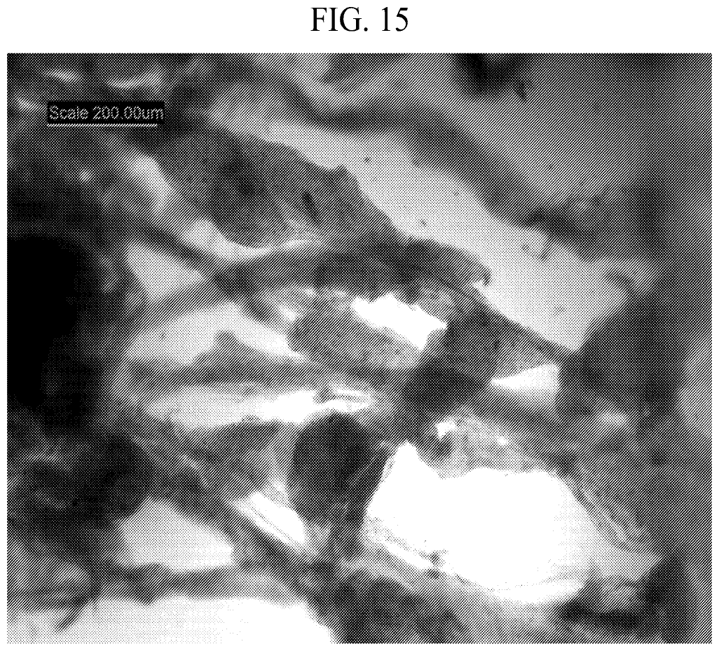

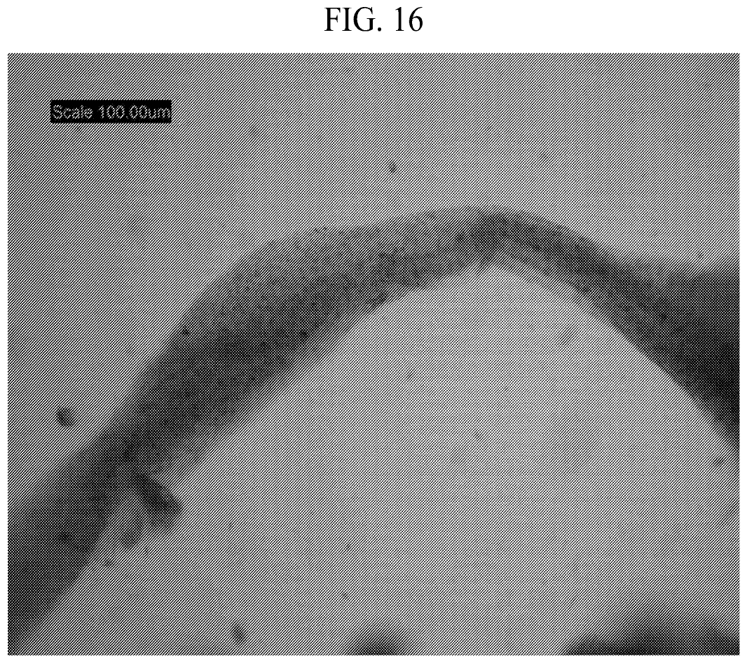

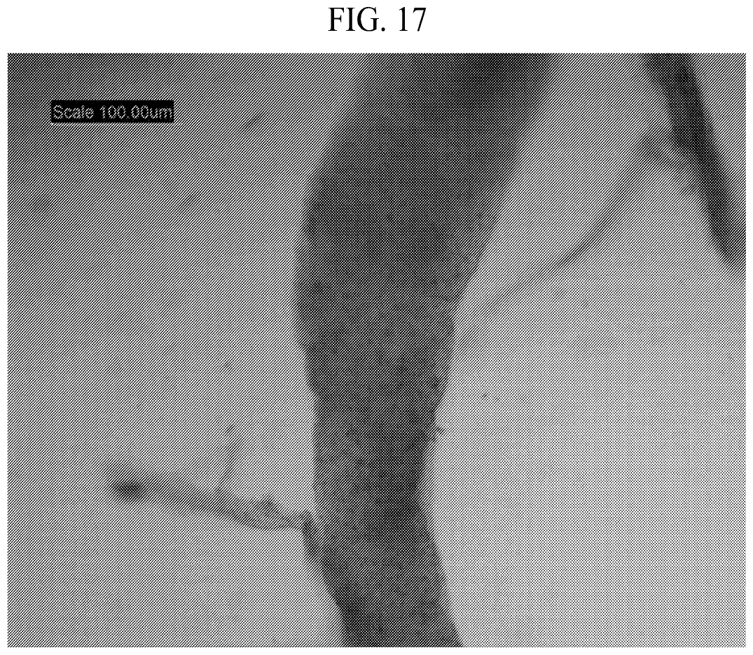

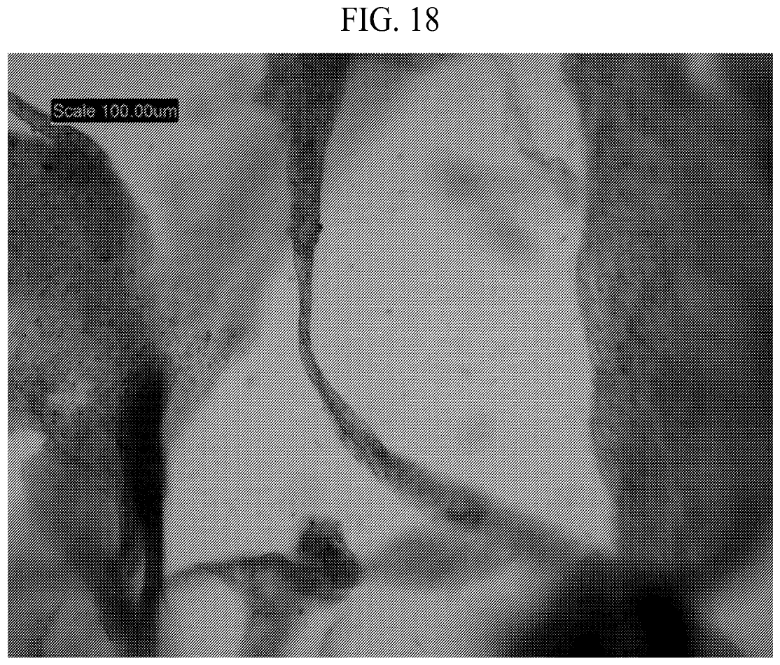

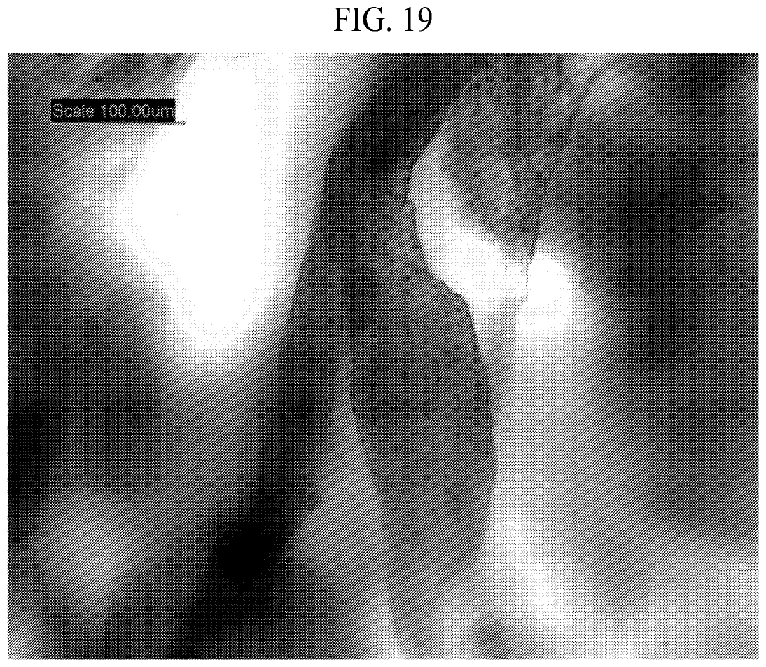

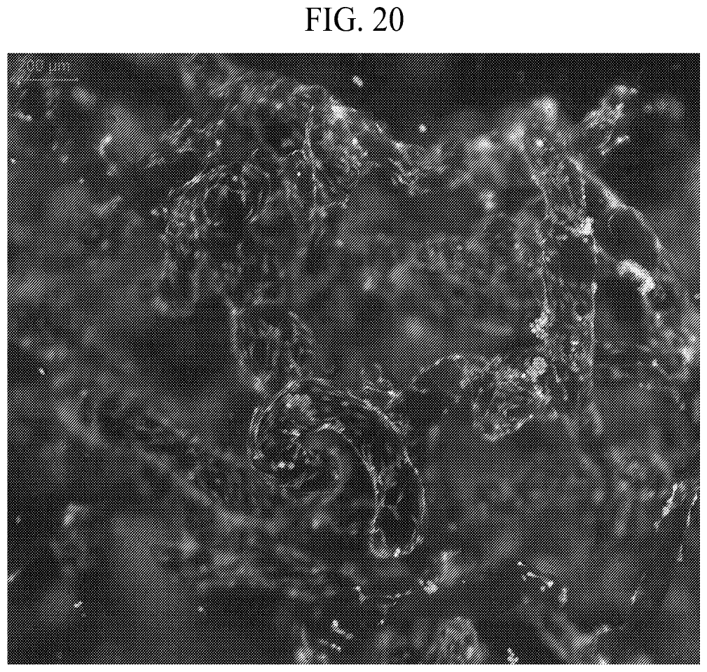

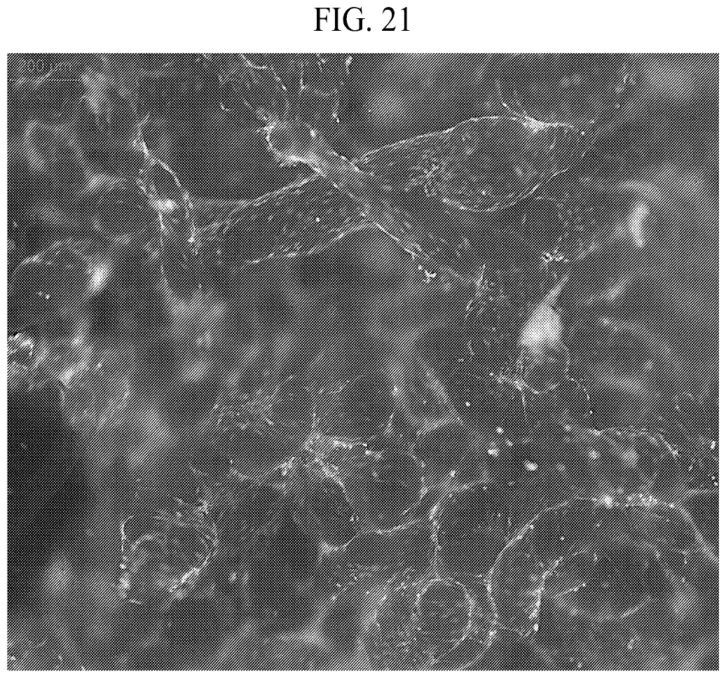

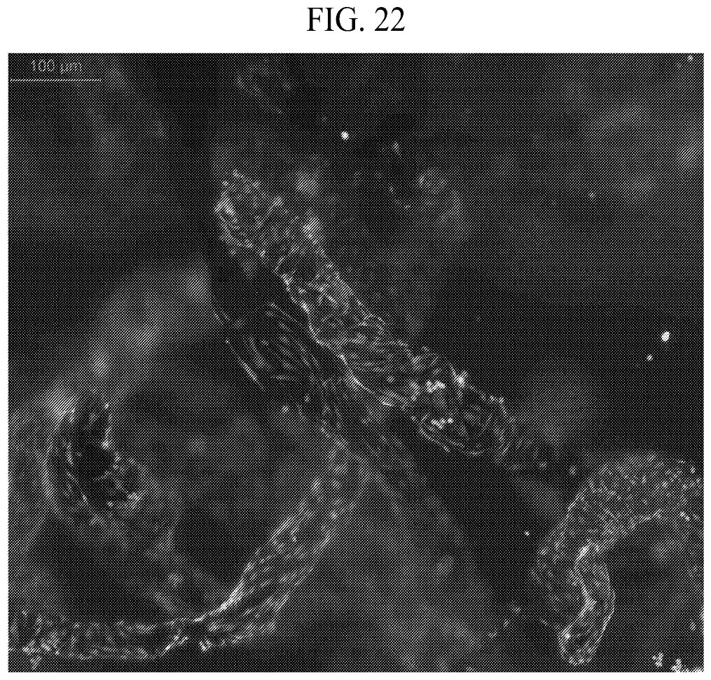

As noted throughout the present disclosure, the scaffolds can, in some embodiments, comprise fibrous scaffolds. FIGS. 10-19 show various SEM and light microscope images of fibrous alginate scaffolds produced via the extrusion techniques described above. Further, FIGS. 10-22 show various fluorescence microscopy images of the fibrous alginate scaffolds to demonstrate the proliferation and growth of cells on the scaffolds. Accordingly, the extruded fibrous scaffolds described herein can be used to culture cells for the manufacture of a cultured meat product.

Additional details regarding structures for cultured meat production can be found in U.S. patent application Ser. No. 16/780,187, published as U.S. Patent Application Pub. No. 2020/0245658, filed Feb. 3, 2020, titled ELECTROSPUN POLYMER FIBERS FOR CULTURED MEAT PRODUCTION, which is hereby incorporated by reference herein in its entirety.

### EXAMPLES

### Example 1: Alginate Electrosprayed Microcarrier Scaffold

In one illustrative example, a scaffold for cultured meat production can be composed of microcarriers formed from a blend of alginate and one or more other plant-based proteins. Alginate is a naturally occurring water-soluble polysaccharide that is present in the cell walls of certain species of brown algae (i.e., Laminaria, Macrocystis, and Ascophyllum). Chemically, alginate is composed of a linear block polymer of β-(1-4)-D-mannuronic acid (M) and α-(1-4)-L-guluronic acid (G). In general, characteristics and functionality of alginate (e.g., gelling capability and gel strength) are affected by the content of these uronic acid polysaccharides with variable molecular weights. Sodium alginate, a salt derivative of alginate, is commonly used in biomedical and industrial applications. It has a strong ability to cross-link and forms a network with water molecules (i.e., a hydrogel) through inter- and intramolecular ionic interactions with divalent ions such as Ca²⁺. Accordingly, alginate hydrogels form three-dimensional networks of hydrophilic polymers with a high water content. In these hydrogels, the guluronate structure of the alginate chains allows a high degree of coordination and binding to divalent cations. This guluronate-ion coordination creates junctions from one polymer chain to the guluronate blocks of an adjacent polymer chain resulting in an ionically cross-linked gel with an egg-box model structure. Alginate is an edible, food grade product.

The other plant-based proteins mixed with the alginate to form the hydrogel microcarrier scaffold can be beneficial for a number of different reasons. For example, incorporating other plant-based proteins into the alginate hydrogel microcarriers can be used to control or modify the physical properties of the scaffold. As another example, incorporating other plant-based proteins into the alginate hydrogel microcarriers can introduce cell-recognition sites to the otherwise hydrophilic alginate surface of the microcarriers, which could enhance the adsorption of serum proteins, particularly for anchorage-dependent cells. As yet another example, incorporating other plant-based proteins into the alginate hydrogel microcarriers can also improve cell adhesion and promote specific cell interactions and other cellular functions (e.g., proliferation, differentiation, and gene expression).

In this specific example, the hydrogel microcarriers can be fabricated via electrospraying. In particular, a stock protein solution was prepared by completely dissolving a known amount of protein powder in water. The solution was heated to 90° C. for 30 min and base-treated with 1 M NaOH to adjust the solution's pH to 9. A stock alginate solution was prepared by completely dissolving a known amount of sodium alginate powder in water. In one implementation, an electrospraying solution containing 1.5% protein and 0.2% alginate was prepared by mixing the stock solutions of protein and alginate (by mass) in water. In another implementation, an electrospraying solution containing 2.21% protein and 0.29% alginate was prepared by mixing the stock solutions of protein and alginate (by mass) in water. Two CaCl₂ solutions (1% and 5%) were prepared by dissolving calcium chloride powder in water.

A 250 ml syringe was filled with an electrospraying solution and was attached to the spinning head of an electrospraying assembly using polyethylene tubing. 25 gauge needles were attached to the multi-emitter spinning head of the I.E-500 machine (Fluidnatek LE-500, Bionicia, Valencia, Spain) and the flow rate for electrospraying was set to 10 ml/hr per needle. The positive and negative voltages used were 30.0 kV and −25.0 kV, respectively. The electrospraying conditions were maintained at 25° C. and 25% relative humidity. A receiving metal container was filled with 5% CaCl₂ solution and the distance between the surface of the liquid and needle tips was set to 10 cm.

The resulting microbeads were soaked overnight in a 1% CaCl₂ solution prior to size separation. In various implementations, surface coating could be performed simultaneously by mixing tannic acid (in 10 mM Tris-HCl solution, pH 8.5) or PDA (in 10 mM Tris-HCl solution, pH 8.5) with the 1% CaCl₂ solution and then soaking the beads for 24 hours. Dry microbeads can be prepared by lyophilization using blast or cryogenic freezing. In other implementations. lyophilized microbeads could be surface-treated via plasma, rather than via tannic acid or PDA. The microbeads then went through a size separation process, which was performed by mechanically sorting the microbeads through different sieve sizes. The microbeads were then sterilized. In various implementations, the microbeads could be sterilized using 70% ethanol, gamma-irradiation, or UV-irradiation treatments. In various implementations, the microbeads could be preserved in a citric acid: calcium chloride solution.

In various implementations of this example, a variety of additives could be incorporated into the protein-alginate electrospraying solution, including food additives, flavoring agents, color additives, preservatives, vitamins, minerals, nutritional additives/enhancers, and cell culture media growth factors. In various embodiments, these additives may be incorporated into the surfaces of the microcarriers by spraying, sprinkling, soaking, or any other surface treatment technique described herein.

FIG. 1 shows an SEM image of a lyophilized microcarrier. This is an image of an electrosprayed microcarrier made from alginate and soy protein isolate that was dried using lyophilization. Lyophilization changes the surface morphology of the microcarries resulting in multiple surface features (i.e., wrinkling) that could facilitate cellular attachment, spreading, orientation, and migration.

FIG. 2 shows an SEM image of a PDA-coated, lyophilized microcarrier. PDA-coated microcarriers provide modified bio-surface with multiple functional groups and motifs that promote cell-adhesion properties, stimulate positive cellular responses, and can render biomimetic and favorable microenvironments that promote cell and tissue functions. Microcarriers with surface properties that promote cell adhesion are especially beneficial for large-scale bioprocessing and production of cultured meat.

FIG. 3 shows an SEM image of a tannic acid-coated, lyophilized microcarrier. Tannic acid coating modifies the surface of microcarrier scaffold which improves cell adhesion, spreading, and long-term culture. Tannic acid-coated scaffolds also result in rapid cell anchorage and cell proliferation rates. The functionalized surface also favors cell spreading and reduces cell apoptosis. Ultimately, similar to PDA-coated scaffolds, tannic acid-coated microcarriers provide good cell adhesion surface properties that is critical for large-scale bioprocessing and production of cultured meat.

FIG. 4 shows a light microscope image (40× magnification) of PDA-coated, non-lyophilized microbeads. This is an image of electrosprayed microcarriers made from alginate and soy protein isolate with uniform diameter (˜200 μm). The black pigmentation on the surface of the microcarriers is due to the PDA treatment.

FIG. 5 shows a light microscope image (40× magnification) of tannic acid-coated, non-lyophilized microbeads. This is an image of electrosprayed microcarriers made from alginate and soy protein isolate. Tannic acid coating results in light tan pigmentation on the surface of the microcarriers.

FIG. 6 shows a light microscope image (100× magnification) of lyophilized (uncoated) microbeads rehydrated in 1× PBS solution. This image shows the rehydration of the lyophilized microcarriers using PBS solution.

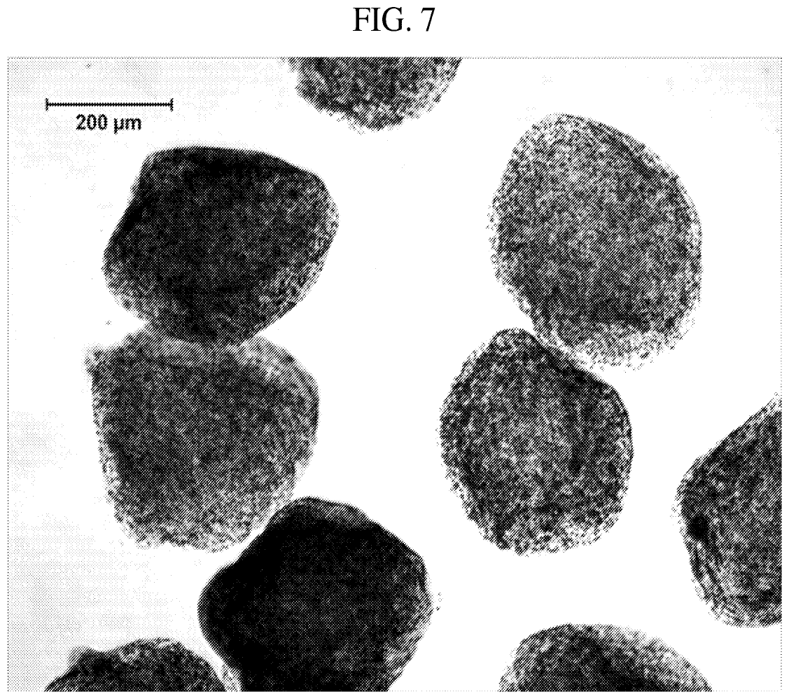

FIG. 7 shows a light microscope image (40× magnification) of PDA-coated, lyophilized microbeads that are rehydrated in 1× PBS solution. This image shows the rehydration of the lyophilized, PDA-coated microcarriers using PBS solution. Retention of the surface characteristics with uniform size upon rehydration are observed due to lyophilization.

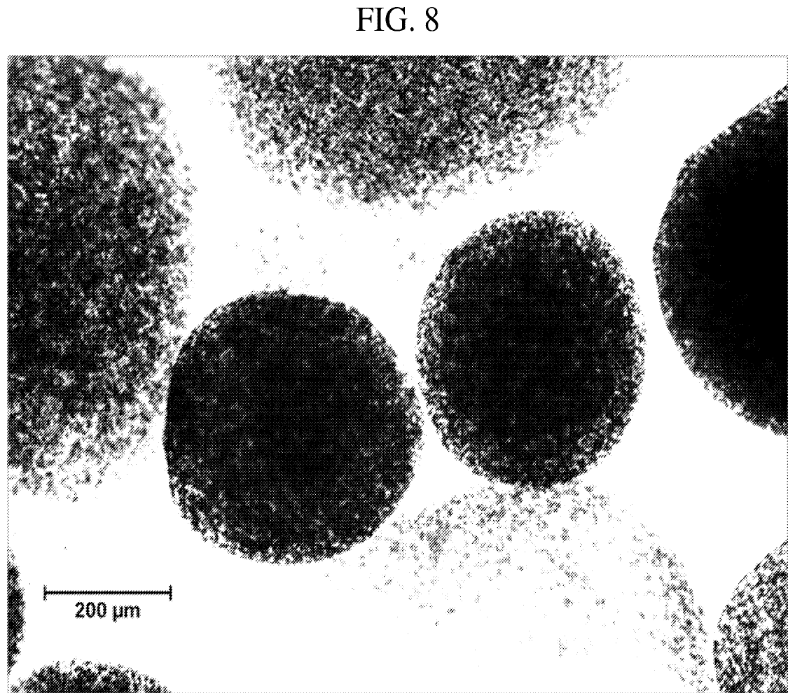

FIG. 8 shows a light microscope image (40× magnification) of tannic acid-coated, lyophilized microbeads that are rehydrated in 1× PBS solution. These tannic acid-coated lyophilized microcarriers regained their spherical shape upon rehydration with PBS.

### Example 2: Alginate Extruded Fibrous Scaffold

In another illustrative example, a fibrous scaffold for cultured meat production can be formed from an extruded blend of alginate and one or more other plant-based proteins. In this illustrative protocol, an alginate precursor gel is prepared. A first solution is formed by mixing 3 wt % soy protein isolate powder into water. The solution can be base-treated to a pH of 9 with 1 M NaOH and heated to 90° C. for 30 minutes. Further, a second solution of 0.4 wt % sodium alginate can be made by dissolving sodium alginate powder in water. The precursor gel can be prepared by mixing the soy and alginate solutions in a 1:1 ratio by mass. Further, a solution of 5 wt % calcium chloride by mass is made for the extrusion bath. Additionally, aqueous solutions of 1 wt % calcium chloride, 10 mM Tris-HCl, and tannic acid are prepared and base-treated to a pH of 8.5 using 1 M NaOH. The concentration of tannic acid can be varied (1 mg/mL, 15 mg/mL, 30 mg/mL, etc.) depending on the specific application for the scaffold.

Further, a syringe with a 27-gauge needle tip is filled with the precursor gel. During extrusion, the needle tip is submerged into a bath of 5% calcium chloride. The fibrous scaffolds are created using an extrusion rate above 360 mL/hour at ambient temperature. Filament formation begins and continues through the entirety of the pumped solution, resulting in semi-continuous solid fibers. When formation of the scaffold is completed, the material is stored in 1% calcium chloride, Tris-HCl, and tannic acid solution for 24 hours for surface coating. The extruded fibers are rinsed in distilled water to remove excess Tris-HCl and tannic acid. This step is repeated with a transition metal cation solution for 24 hours and rinsed in a similar fashion. FIGS. 10-19 show various SEM and light microscope images of scaffolds produced using the techniques described herein. Further, FIGS. 10-22 show various fluorescence microscopy images of the scaffolds produced using the techniques described herein to demonstrate the proliferation and growth of cells on the scaffolds, demonstrating the ability of these types of scaffolds to be used for cultured meat production.

### Example 3: Surface Modification Treatment Technique for Edible Scaffolds

As generally noted above, the scaffold structures can be treated with a variety of different surface modification treatments in order to enhance the growth of the cells thereon. In one implementation, a microbead scaffold (e.g., formed via electrospraying) can undergo the application of both a tannic acid coating and a cationic transition metal. In particular, the microbead scaffold can be treated with 15 mg/ml tannic acid in water for a period of 24 hours. The tannic acid-treated microbead scaffold can then be treated with 1 mg/ml iron (III) citrate in water for a period of 4 hours. Accordingly, the microbead scaffold can include both a tannic acid coating and a cationic transition metal surface modification treatment.

While the present disclosure has been illustrated by the description of exemplary embodiments thereof, and while the embodiments have been described in certain detail, it is not the intention of the Applicants to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

1. An edible scaffold for cultured meat production, the scaffold comprising:
a structural element comprising a polysaccharide and a plant-based protein, wherein the structural element comprise a diameter between about 1 μm to about 1000 μm;
wherein the structural element has been formed via extrusion or an electrohydrodynamic technique from a solution comprising the polysaccharide and the plant-based protein;
wherein the structural element has undergone a surface modification treatment configured to facilitate attachment by cells, the surface modification treatment comprises at least one of a plant-derived polyphenolic compound, a cationic transition metal, or a plasma treatment;
wherein the scaffold is formed in a shape configured to produce a meat product having a dimension from about 100 μm to about 500 mm when the cells are cultured thereon.

2. The edible scaffold of claim 1, wherein the structural element comprises a continuous fiber network.

3. The edible scaffold of claim 1, wherein the structural element comprises microcarriers.

4. The edible scaffold of claim 1, wherein the polysaccharide is configured to form a hydrogel.

5. The edible scaffold of claim 1, wherein the polysaccharide comprises alginate.

6. The edible scaffold of claim 1, wherein the electrohydrodynamic technique comprises at least one of electrospraying or electrospinning.

7. The edible scaffold of claim 1, wherein the polysaccharide and the plant-based protein are present in a ratio from about 100:1 to about 1:20 by dry weight.

8. The edible scaffold of claim 1, wherein the structural element is lyophilized.

9. (canceled)

10. The edible scaffold of claim 1, wherein the plant-based protein comprises at least one of a pea-based protein, soy-based protein, oat-based protein, mung bean-based protein, maize-based protein, chia-based protein, hemp protein, prolamin, pumpkin seed-based protein, rice-based protein, sunflower seed-based protein, or sacha inchi-based protein.

11. The edible scaffold of claim 1, further comprising at least one of a food additive, a flavoring agent, a color additive, a preservative, a vitamin, a mineral, a nutritional additive or enhancer, a synthetic protein, a peptide, a ligand, or a cell culture media growth factor.

12. The edible scaffold of claim 1, wherein the structural element is configured to undergo syneresis in response to a trigger.

13. The edible scaffold of claim 12, wherein the trigger comprises a calcium chelator.

14. (canceled)

15. The edible scaffold of claim 1, wherein the surface modification treatment comprises iron citrate.

16. A method of producing an edible scaffold for cultured meat production, the method comprising:
depositing a solution comprising a polysaccharide and a plant-based protein into a cross-linking bath to form the edible scaffold comprising a structural element, the structural element comprising a polysaccharide and a plant-based protein, wherein the structural element comprise a diameter between about 1 μm to about 1000 μm;
applying a surface modification treatment to the scaffold, the surface modification treatment configured to facilitate attachment by cells, the surface modification treatment comprises at least one of a plant-derived polyphenolic compound, a cationic transition metal, or a plasma treatment;
wherein the scaffold is formed in a shape configured to produce a meat product having a dimension from about 100 μm to about 500 mm when the cells are cultured thereon.

17. The method of claim 16, wherein depositing the solution comprises extruding the solution.

18. The method of claim 16, wherein depositing the solution comprises at least one of electrospraying or electrospinning the solution.

19. The method of claim 16, wherein the structural element comprises a continuous fiber network.

20. The method of claim 16, wherein the structural element comprises microcarriers.

21. The method of claim 16, wherein the polysaccharide is configured to form a hydrogel.

22. The method of claim 16, wherein the polysaccharide comprises alginate.

23. The method of claim 16, wherein the polysaccharide and the plant-based protein are present in a ratio from about 100:1 to about 1:20 by dry weight.

24. The method of claim 16, further comprising lyophilizing the edible scaffold.

25. (canceled)

26. The method of claim 16, wherein the plant-based protein comprises at least one of a pea-based protein, soy-based protein, oat-based protein, mung bean-based protein, maize-based protein, chia-based protein, hemp protein, prolamin, pumpkin seed-based protein, rice-based protein, sunflower seed-based protein, or sacha inchi-based protein.

27. The method of claim 16, the edible scaffold further comprises at least one of a food additive, a flavoring agent, a color additive, a preservative, a vitamin, a mineral, a nutritional additive or enhancer, a synthetic protein, a peptide, a ligand, or a cell culture media growth factor.

28. The method of claim 16, wherein the structural element is configured to undergo syneresis in response to a trigger.

29. The method of claim 28, wherein the trigger comprises a calcium chelator.

30. (canceled)

31. The method of claim 16, wherein the surface modification treatment comprises iron citrate.
